(19) United States
(12) Patent Application Publication
(10) Pub. No.: US 2006/0257909 A1
(43) Pub. Date: Nov. 16, 2006

(54) Methods for reducing the percentage of abnormal gametes

(76) Inventors: Gary Harton, Manassas, VA (US); David Karabinus, Manassas, VA (US); Harvey Stern, Olney, MD (US)

Correspondence Address: MCDERMOTT WILL & EMERY LLP, 600 13TH STREET, N.W., WASHINGTON, DC, 20005-3096, US

(21) Appl. No.: 11/413,123

(22) Filed: Apr. 28, 2006

Related U.S. Application Data
(60) Provisional application No. 60/675,462, filed on Apr. 28, 2005.

(51) Int. Cl. C12Q 1/68

(52) U.S. Cl. 435/6

(57) ABSTRACT

The invention relates to methods of enriching a sperm sample for euploid sperm comprising obtaining a sperm sample from a mammalian male; contacting the sperm cells with a detectable DNA-interacting agent that imparts a sperm cell with a signal intensity proportional to the amount of DNA present in the sperm cell; and separating those sperm cells that have a signal intensity indicative of euploid sperm from those that have a signal intensity indicative of aneuploid sperm.

### PRIORITY STATMENT

The application claims priority to U.S. Ser. No. 60/675,462 filed Apr. 28, 2005, which is incorporated by reference in its entirety for all purposes.

### BACKGROUND OF THE INVENTION

1. Field of the Invention

This invention relates to a method of enriching a sperm sample for healthy spermatozoa by sorting sperm based on differences in DNA content.

2. Background

There are many causes of infertility or recurrent pregnancy loss in humans. One etiology involves structural rearrangements of the chromosomes (e.g., translocations or inversions) which result in abnormal embryos and recurrent miscarriage or apparent infertility. In other cases, males may produce sperm with an increased incidence of numerical chromosomal abnormalities, e.g., germinal mosaicism, which can result in non-viable embryos with missing or extra chromosomes.

Approximately one in 350 individuals carries either a balanced reciprocal or a Robertsonian (whole arm) translocation (1.6/1000 population frequency for reciprocal translocation and 1.2/1000 for Robertsonian translocations). Men who are translocation carriers are physically normal, but can produce abnormal gametes sperm). These abnormal gametes can result in multiple pregnancy losses, apparent infertility, or, in some cases, birth of children with mental and/or physical disabilities. Cytogenetic analysis of sperm in male carriers of translocations has revealed a wide variability in the percentage of sperm that is abnormal, ranging from 1% or less to as high as 77% (Martin et al. 1995, Escudero et al. 2002).

Preimplantation genetic diagnosis (PGD) has been performed on embryos from couples where one partner carries a translocation, resulting in a significant improvement in the pregnancy rate and a dramatic lowering of the loss rate when chromosomally appropriate (i.e., normal or balanced) embryos are identified after analysis by multi-color fluorescence in-situ hybridization (FISH). Previous work (Munne, et al. 2002) with couples where the male is the translocation carrier has shown a correlation between the percentage of chromosomally abnormal sperm and the yield of chromosomally appropriate embryos (the higher the percentage abnormal sperm, the fewer normal/balanced embryos were seen following PGD). When the percentage of abnormal sperm increases to around 65%, no normal embryos are identified by PGD. This latter fact directly correlates with the ability to achieve pregnancy in assisted reproductive technology (ART) cycles for couples where one partner carries a translocations and PGD is used to identify translocations.

Recent studies have examined the recurrence risk of trisomic conceptions after a pregnancy history of a trisomic fetus. It has been concluded that the observed recurrence was too high to be caused by chance events and that some couples may have a predilection for producing abnormal gametes, which result in chromosomally abnormal embryos. Germinal mosaicism refers to a situation where chromosome abnormalities exist in at least a part of the gametes but not in any other cells in the body. The individual is therefore physically normal, but can produce high rates of chromosomally abnormal gametes, embryos and fetuses. This phenomenon has been demonstrated in men due to the availability of gametes for study. Such men will have at least two populations of sperm, one of which contains an extra or missing chromosome resulting in aneuploid conceptions.

The X chromosome is larger and contains more DNA than does the Y chromosome. The difference in total DNA content between X-bearing sperm and Y-bearing sperm is 3.4% in boar, 3.8% in bull, 4.2% in ram and 2.8% in human sperm. The amount of DNA in a normal sperm cell, as in most normal body cells, is stable. The DNA content of individual sperm, therefore, can be monitored and used to differentiate X- and Y-bearing sperm. Fugger et al., (Hum Reprod, Vol. 13, No. 9, 1998, pp. 2367-2370), describe the first births and clinical trial data from the use of MicroSort® technology to separate X and Y sperm by flow cytometric cell sorting followed by intrauterine insemination (IUI), in vitro fertilization (IVF), or intracytoplasmic sperm injection (ICSI). The reference discloses that the separation of human X- and Y-bearing sperm cells is based on the 2.8% difference in total DNA content between the sperm populations.

Vidal et al., (Human Reproduction, December 1999, Vol. 14, No. 12, 2987-2990), describe the use of fluorescence in-situ hybridization (FISH) to evaluate the viability and rate of aneuploidy in sperm following flow cytometry sorting (FCS) to separate human X- and Y-bearing spermatozoa, based on the difference in their DNA content. Vidal reported of an excess of Y-bearing spermatozoa among those spermatozoa disomic for chromosome 21, which suggested that the extra chromosome 21 preferentially segregates with the Y chromosome. Vidal calculates that sperm with two (instead of the normal 1) X-chromosomes (XX disomies) have a 9% excess in total DNA content, whereas spermatozoa disomic for chromosome 21 have a 1.5% excess in total DNA content compared to wildtype (i.e., normal) sperm.

The Inventors have discovered a method for using fluorescence-activated cell sorting to identify and separate human sperm based upon sperm DNA content (MicroSort®) to enrich a sperm sample with gametes containing chromosomally appropriate (normal/balanced) segregants. The method has the ability to increase the percentage of chromosomally appropriate sperm in a sample which can lead to an improvement in the outcome of in vitro fertilization (IVF) cycles in terms of producing a greater number of embryos available for transfer following PGD.

All publications, scientific, patent or otherwise are hereby incorporated by reference in their entirety for all purposes. Particularly, Martin et al., Clinical Genetics 1995: 47: 42-46. Escudero et al., Prenatal Diagnosis 2002: 20: 599-602. Munné et al., Reproductive Biomedicine Online (RBM Online) 2002a: vol. 4. no. 2. 183-196. Munné et al., Abstract Presented at the 58th Annual Meeting of the American Society for Reproductive Medicine (ASRM), 2002, Seattle, Wash.; and Daniel, Human Genet 51; 171-182:1979.

### SUMMARY OF THE INVENTION

One aspect of the invention relates to a method of enriching a sperm sample for euploid sperm comprising obtaining a sperm sample comprising sperm cells from a mammalian male; contacting the sperm cells with a detectable DNA-interacting agent that imparts a sperm cell with a signal intensity proportional to the amount of DNA present in the sperm cell; separating those sperm cells that have a signal intensity indicative of euploid sperm from those that have a signal intensity indicative of aneuploid sperm; and collecting the separated sperm cells to obtain an enriched sperm sample.

In one embodiment, the agent is Hoechst 33342. In another embodiment, the separating is carried out by flow cytometery. In yet another embodiment, the flow cytometery is fluorescence activated cell sorting. In still another embodiment, the signal intensity indicative of euploid sperm corresponds to a sperm karyotype index between about 99.5 and about 103; wherein a sperm karyotype index of 100 is defined as the signal intensity associated with normal Y-bearing sperm and a sperm karyotype index of 102.8 is defined as the signal intensity associated with normal X-bearing sperm. In a further embodiment, the sperm sample and the enriched sperm sample are analyzed by fluorescence in situ hybridization (FISH) for chromosomal abnormalities. In still a further embodiment, the male produces sperm that contain structural chromosomal abnormalities. In yet another embodiment, the aneuploid sperm have numerical chromosomal abnormalities. In another embodiment, the male is a translocation carrier. In yet another embodiment, the male is selected from the group consisting of a human, a bull, a goat, and a boar.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the drawings and description are to be regarded as illustrative in nature, and not as restrictive.

### DETAILED DESCRIPTION OF THE INVENTION

This invention relates to a method of enriching a sperm sample from a male with a structural rearrangement of the sperm chromosomal DNA or with numerical abnormalities of sperm chromosomes. The goal is to increase the proportion of competent spermatozoa by sorting sperm based on differences in DNA content. This purified sperm can be used to fertilize the partner's eggs by intrauterine insemination (IUI), in vitro fertilization (IVF), or intracytoplasmic sperm injection (ICSI) to hopefully produce healthy offspring. However, before describing the invention in greater detail the following terms are defined:

“Enriching a sperm sample for euploid sperm” refers to the aim of the invention to increase the proportion of genetically normal spermatozoa in a sperm sample. Euploid spermatozoa contain the genetic attributes necessary for the development of a healthy zygote, embryo, fetus and ultimately the birth of a normal and healthy baby. Euploid sperm cells that suffer from decreased motility or life span may still be used to fertilize an oocyte using such techniques as microinjection, e.g., intracytoplasmic sperm injection (ICSI). Alternatively, aneuploid sperm are those sperm that lack the genetic integrity to give rise to a medically normal and healthy baby.

Human cells normally possess 46 chromosomes comprising 22 pairs of autosomes (numbered 1 to 22) and 2 sex chromosomes (X and Y). Gametes (egg and sperm) each contribute one-half of the genetic material, or one member of each of the autosome pairs and one sex chromosome, to the embryo. Each gamete contains 23 chromosomes. The process, which results in gametes having half the genetic material of the somatic cells, meiosis, requires an even, symmetrical distribution of genetic material in dividing cells. A disruption in meiosis can result in an asymmetric gameteic chromosome compliment, as occurs in gametes of individuals who carry structural rearrangements of the chromosomes or a greater or lesser than the normal haploid (23) number of chromosomes. Regardless of their motility or life span, aneupolid sperm contain chromosomal abnormalities that often result in too much or too little DNA often times resulting from abnormal segregation of chromosomes during meiosis. Examples of such chromosomal abnormalities are numerical abnormalities due to non-disjunction, deletions, and translocations.

Numerical aneuploidy is a result of chromosomal nondisjunction. Nondisjunction is the failure of homologous chromosomes or sister chromatids to separate or segregate at meiosis or mitosis, such that one daughter cell has 2 chromosomes or chromatids and the other has none. For sperm, this means that a given sperm cell may have two copies of a particular chromosome while another sperm of that cohort may have no copy of that chromosome. Conceptions from these sperm will result in embryos with extra or missing chromosomes.

Reciprocal translocations are the most common structural chromosomal rearrangement in humans and result from the two-way exchange of genetic material between non-identical (non-homologous) chromosomes. This process involves the movement of a segment of a chromosome from its normal location to a new site on another chromosome creating two derivative chromosomes. Translocations occur when segments of the two involved chromosomes become physically apposed, with a break and exchange of material occurring at regions where localized homology occurs in the form of recurrent or repeated DNA sequences in non-coding regions of the genome. The chromosome segment that is exchanged is called the translocated segment, and the remainder of the chromosome including the centromere or anchoring region is called the centric segment.

Individuals who carry reciprocal translocations are generally healthy. However, during the meiotic phases of gametogenesis, they are prone to producing chromosomally unbalanced and, therefore, aneuploid sperm or eggs. Many embryos formed from unbalanced gametes either fail to implant, or may result in early, pre-clinical pregnancy loss. Unfortunately, some pregnancies from translocation carriers may progress further in gestation and result in a spontaneous miscarriage or an abnormal fetus discovered at prenatal diagnosis or even at birth. The individual outcomes are generally dependent on the chromosomes involved as well as the size and segregation pattern of the translocation. The degree of aneuploidy often impacts the embryo's capability to undergo further development. In many cases, the pregnancy loss rate when one of the partners carries a balanced translocation is approximately 80%. Frequently, the reproductive history involves 5-9 pregnancy losses with the birth of an occasional normal (or abnormal) child.

Approximately one in 350 individuals carry either a balanced reciprocal or Robertsonian (whole arm) translocation (1.6/1000 population frequency for reciprocal translocation and 1.2/1000 or Robertsonian translocation). Individuals who are translocation carriers are phenotypically normal but can produce abnormal gametes (eggs and sperm). These abnormal gametes can cause multiple pregnancy losses, apparent infertility, or in some cases, birth of children with mental and/or physical disabilities. Cytogenetic analysis of sperm in male carriers of translocations has revealed a wide variability in the percentage of sperm that is abnormal, ranging from 1% or less to as high as 77%. Previous work in couples where the male is the translocation carrier has shown a correlation between the percent of sperm that is chromosomally abnormal and the yield of chromosomally appropriate embryos (the higher the percentage abnormal sperm, the fewer normal/balanced embryos were seen following PGD). Escudero (2002) has reported that men with >65% abnormal sperm segregants rarely produce chromosomally appropriate embryos by IVF.

Robertsonian translocations involve the movement of the entire long arm of the acrocentric chromosome with exchange in the short arm resulting in a dicentric chromosome with loss of most of the short arms of both chromosomes. These consist mostly of repetitive DNA and ribosomal RNA genes, which are repeated in the other acrocentric chromosomes. The loss of the short arms of the translocated chromosome is therefore not pathologic. Robertsonian translocation carriers, however, have 45 chromosomes including a derivative chromosome resulting from fusion of two chromosomes which may be the same (homologous i.e., 13; 13 Robertsonian translocation) or different (non-homologous i.e., 13; 21 Robertsonian translocation). For the carrier of a Robertsonian translocation the possible gametes at meiosis depend on the segregation of the fused derivative chromosome and the two other single chromosomes. For illustration, consider the common chromosome 13; 14 Robertsonian translocation. A gamete may be normal (containing a single copy of a chromosome) if the derivative chromosome is not included, but the normal 13 and 14 chromosomes are present. Alternatively, the gamete is balanced (and normal) if only the derivative 13; 14 chromosome is present. In each case, a single copy of both chromosomes 13 and 14 is present. If, however, the derivative chromosome is included with either of the normal 13 or 14 chromosome, a disomic (two copy) gamete is produced that when fertilized, can lead to an embryo with trisomy for either chromosome 13 or 14. Alternatively, gametes can be produced which do not contain either the 13; 14 derivative or the normal 13 or 14 chromosomes. This leads to a nullisomic (no copy) gamete, which can produce a monosomic (one copy) embryo. What all these situations have in common is that there is a disturbance in the amount of DNA present in the gamete from the normal haploid content (i.e. one copy of each chromosome).

“Contacting the sperm cells with a detectable DNA-interacting agent” refers to the association of the sperm cells of the sperm sample with an agent that interacts with the DNA in the sperm cells. Preferably the agent is a membrane permiant, noncytotoxic, supravital DNA specific fluorochrome. The interaction between the agent and DNA, can take place through ionic, covalent or hydrogen binding, for example, so long as the genetic health of the cell is preserved. Preferably, the more DNA in the cell, the more DNA-interacting agent becomes associated with the cell. Even more preferably, a greater amount of DNA in a sperm cell results in the association with a greater amount of detectable DNA-interacting agent and a corresponding increase in detectable signal, e.g. fluorescence, emanating from the cell. In the preferred embodiment, the detectable DNA-interacting agent is a fluorescent DNA dye that is suitable for use in flow cytometry applications. Preferably, the fluorescent agent is selected from the group consisting of, but not limited to, Hoechst 33342, DAPI, Hoechst 33258, SYTOX Blue, Chromomycin A3, Mithramycin, YOYO-1, SYTOX Green, SYTOX Orange, Ethidium Bromide, 7-AAD, Acridine Orange, TOTO-1, TO-PRO-1, Thiazole Orange, Propidium Iodide (PI), TOTO-3, TO-PRO-3 and LDS 751. Most preferably, the fluorescent agent is Hoechst 33342.

The term “separating those sperm cells that have an activity intensity associated with euploid sperm” refers to the differentiation of euploid and aneuploid sperm on the basis of differences in DNA content between the two sperm cell classes. The sperm cells within a sample are separated by a device that can determine whether the accumulated activity, i.e., activity intensity, of a sperm cell that has been contacted with a detectable DNA-interacting agent, is associated with aneuploidy or euploidy.

Preferably, the activity intensity is an amount of fluorescence directly related to the amount of DNA in the sperm cell. Preferably, a flow cytometry apparatus separates cells that have a given range, i.e., window of fluorescence. Setting up a flow cytometer to separate cells in a particular window of fluorescence is also referred to as “gating” the flow cytometer. Accordingly, the invention envisages calcsating a window or fluorescence that is indicative of euploid sperm.

For example, the separation of X- from Y-chromosome bearing sperm is based on the fact that human Y-bearing sperm contain about 2.8% less DNA than X-bearing sperm. In a similar fashion, the unbalanced gametes of a male carrying a 13; 14 Robertsonian translocation contains differing amounts of DNA, and can thus be identified and sorted. One can determine the quantitative amount of a particular segmental imbalance as a fraction of the entire haploid genome or haploid autosomal length (HAL). For chromosome 13 this is 3.74% and chromosome 14, 3.56%. In this invention, the lower of the two values was used as to be overly exclusive of aneuploid sperm. As such, both chromosome 13 and 14 will be set to have a HAL of about 3.5%. One can, therefore, predict the differences in DNA content of the possible segregants. In each case there will be male (Y-bearing) and female (X-bearing) sperm cells that will differ by about 2.8%.

The term “sperm karyotype index” refers to the DNA content of a spermatozoa relative to a single 23, Y (normal, Y-bearing sperm). Under this system, the DNA content of a single 23, Y is assigned the value 100. By extension, an X-bearing sperm will have a value of about 102.8. In a similar fashion, one can generate the expected DNA content for the possible segregants of a male carrying a Robertsonian 13:14 translocation as well as non-disjunction of the 21 chromosome which has a HAL of 1.5%. See Table 1.

TABLE1

| | Male | Female |
|---|---|---|
| Karyotype of Sperm | sperm karyotype index | sperm karyotype index |
| 23, Normal/Balanced | 100 | 102.8 |
| 24, disomy 13 | 103.5 | 106.3 |
| 24, disomy 14 | 103.5 | 106.3 |
| 22, nullisomy 13 | 96.5 | 99.3 |
| 22, nullisomy 14 | 96.5 | 99.3 |
| 22, nullisomy 21 | 98.5 | 101.3 |
| 24, disomy 21 | 101.5 | 104.3 |

By examining the chromosomal distribution of the expected segregants it is possible to set “windows” for collecting sperm on the flow-activated cell sorter. In the above example, the isolating of sperm with sperm karyotype index of approximately 100 (Y-bearing sperm) would be expected to result in a relative increase in the percentage of chromosomally normal sperm. Preferably, flow cytometry enriches a sperm sample for euploid sperm by separating those sperm that have a karyotic index of about 99.5 to about 103 from sperm that have a karyotypic index below about 99.5 and above about 103. In this example, the “window” is a sperm karyotypic index of about 99.5 to about 103.

Spermatozoa having a sperm karyotypic index outside the window are classified to be aneuploid because they have either too little or to much DNA. One of skill in the art will recognize that when a male is known carrier of a particular translocation, the window may be adjusted to maximize the amount euploid sperm to be isolated based on calculations of increasing or decreasing DNA content relative to normal Y-bearing sperm, based on segregation analysis. Additionally, the window could be adjusted to take into account differences in DNA between X and Y chromosomes of sperm from different species such as pig, goat, horse, bull, canine, feline, etc. For example, to separate euploid from aneuploid boar sperm a karyotypic index window of about 99.5 to about 104. For bull sperm, a karotyic index window of about 99.5 to about 104.5 and for ram sperm a karotyic index window of about 99.5 to about 104.5, is preferable.

It is important to note that one will not be able to eliminate all chromosomally abnormal sperm, but the goal is to decrease the percentage of sperm with chromosome abnormalities in a sample. In a similar manner, this approach can be applied to chromosomal anomoly where the segregants result in a significant difference of DNA content of the sperm, which can be detected on flow activated cell sorting.

For small differences in DNA to be detected between euploid and aneuploid sperm, the sperm is subjected to flow cytometry. Details of the preferred generalized methodology of sperm flow cytometry are described in U.S. Pat. Nos. 5,985,216 and 5,135,759, which are hereby incorporated by reference in their entirety. Preferably, sperm passes single file through the laser beam, which measures the DNA content of individual sperm by way of its association with the detectable DNA specific agent. Most preferably, in orthogonal flow cytometry, a suspension of single cells stained with a fluorochrome is made to flow in a narrow stream intersecting an excitation source (laser beam). As single cells pass through the beam, optical detectors collect the emitted light, convert the light to electrical signals, and the electrical signals are analyzed by a multichannel analyzer. The data are displayed as multi- or single-parameter histograms, using number of cells and fluorescence per cell as the coordinates.

In order to use an orthogonal flow cytrometric system to differentiate between euploid and aneuploid sperm DNA, a standard sample injection tip, a beveled sample injection tip and or a specially modified orienting nozzle and second fluorescence detector in the forward position may be employed. Preferably, the modified system allows one to control the orientation of the flat ovoid sperm head as it passes the laser beam. Elimination of the unoriented sperm by electronic gating enhances precision and efficiency. Typically, about 80% of sperm nuclei (without tails) are properly oriented as they pass the laser beam.

Most preferably, in a modified BD Vantage® SE flow cytometer/cell sorter, hydrodynamic forces exerted on the flat, ovoid mammalian sperm nuclei orient the nuclei in the plane of the sample stream as they exit the injection tip. Fluorescent signals may then be collected simultaneously by 90 and 0 degree optical detectors, from the edge and flat side of the sperm nucleus, respectively. For sorting, the sample stream is broken into uniform droplets by an ultrasonic transducer. Individual droplets containing single sperm of the appropriate fluorescence intensity are given a charge and electrostatically deflected into collection vessels. The collected sperm nuclei then can be used for microinjection, e.g., by intracytoplasmic sperm injection (ICSI), into eggs. Since the sperm nuclei have no tails, they cannot be used for normal insemination.

Accurate measurement of mammalian sperm DNA content using flow cytometry and cell sorting is difficult because the sperm nucleus is highly condensed and flat in shape, which makes stoichiometric staining difficult and causes stained nuclei to have a high index of refraction. These factors contribute to emission of fluorescence preferentially from the edge or thin plane of the sperm nucleus. In most flow cytometers and sorters, the direction of sample flow is orthogonal to the direction of propagation of the laser beam and the optical axes of the fluorescence detection. Consequently, fluorescence measurement is most accurate when the fluorescent stain in sperm nuclei is excited and the fluorescence is measured on an axis perpendicular to the plane of the sperm head. At relatively low sample flow rates, hydrodynamics are used to orient tailless sperm so that DNA content can be measured precisely on about 60 to 80% of the sperm passing in front of the laser beam. The preferred modified BD Vantages® system can measure the DNA content of tailless sperm from most species at the rate of about 50 to about 150 sperm per second.

Intact sperm (with tails), whether viable or nonviable, cannot be oriented as effectively as tailless sperm nuclei. However, a 90-degree detector can be used to select the population of properly oriented intact sperm to be measured by the 0 degree detector. Since no hydrodynamic orientation is attempted, the sample flow rate can be much higher, which compensates somewhat for the fact that only about 15 to 20% of intact sperm pass through the laser beam with proper orientation. In this invention, the overall flow rate is approximately 2500 or more intact sperm per second. The intact euploid and aneuploid sperm fractions are sorted simultaneously from the population of input sperm at a rate of about 80-90 sperm of each type per second.

It is, of course, of critical importance to maintain high viability of the intact sperm during the sorting process and during storage after sorting but prior to insemination. Of the factors involved in maintaining sperm viability, the method of staining, the sheath fluid, and the collecting fluid have been found to be especially important.

A nontoxic detectable DNA-interacting agent must be selected. A preferred stain is Hoechst bisbenzimide H 33342 fluorochrome (Calbiochem-Behring Co., La Jolla, Calif.). Preferably, concentration of the fluorochrome is be minimal to avoid toxicity, and yet be sufficient to stain sperm uniformly and to detect the small differences in the DNA of euploid and aneuploid sperm with minimal variation. A suitable concentration was found to be 5 μg/ml, but this may be varied from 4 to 5 μg/ml.

Preferably, the sperm sample is incubated with stain at sufficient temperature and time for staining to take place, but under mild enough conditions to preserve viability. Incubation for 1 hr at 35° C. was found to be acceptable, but ranges of 30° to 39° C. would also be effective. Incubation time should be adjusted according to temperature; e.g., 1.5 hr for 30° C.; 1 hr for 39° C.

Sheath fluid used in sorting cells should be electrically conductive and isotonic and compatable with maintaining cell viability. A concentration of about 10 mM phosphate buffered saline provides the preferred electrical properties, and about 0.1% bovine serum albumin may be added to enhance sperm viability by providing protein support for metabolism and viscosity for the sperm. Preferably, the sheath fluid is free of sugars and excess salts.

Dilution of sperm as occurs in sorting tends to reduce viability of the cells. To overcome this problem, sperm may be collected in test egg yolk extender (TYB) that may be modified by adjusting the pH and/or by adding a surfactant. Details of the composition of the extender are described in U.S. Pat. No. 5,136,759, which is hereby incorporated by reference in its entirety. The surfactant is believed to enhance capacitation of the sperm prior to fertilization.

To confirm the DNA content and enhanced overall competence of a post-sorted sperm sample, an aliquot of the sorted sperm may be analyzed for the presence of chromosomal anomalies. Such analysis may include but not be limited to Polymerase Chain Reaction (PCR) analysis or fluorescence in situ hybridization (FISH) analysis. Preferably, FISH testing of sperm using translocation—specific probes is used to determine the percentage of normal and abnormal segregants in a sperm sample before and after flow cytometry. Most preferably, the flow cytometry procedure allows a strategy to be developed to decrease the percentage of abnormal aneuploid sperm in the post-sorted sample relative to the amount in the pre-sorted sample.

### EXAMPLE 1

Sperm Preparation and Staining

The study participant provided fresh semen for sorting. Prior to evaluation and processing, freshly collected semen was allowed to liquefy at 35° C. for 30 minutes. All semen was evaluated for volume, concentration, percentage motile, progression (grade 0-3), and viability (eosin dye exclusion) before and after processing. Semen was processed to recover motile sperm and to remove undesirable seminal components by discontinuous density gradients (ISolate, 50%, 90%, Irvine Scientific, Santa Ana, Calif.). After processing, recovered sperm were washed and the pellets resuspended in BWW (Irvine Scientific, Santa Ana, Calif.) supplemented with 10% bovine serum albumen (BA; Sigma, St Louis, Mo.), and then stained for 1 hour at 37° C. with Hoechst 33342 (Calbiochem-Behring Corporation, La Jolla, Calif.) at a final concentration of 9 μM as previously described (Johnson et al., Hum. Reprod., 8, 1733-1739, 1993).

### EXAMPLE 2

Flow Cytometric Separation

Stained sperm may be sorted using either a modified Epics® 753 (Coulter Corporation, Hialeah, Fla.) or modified FACS® Vantage flow cytometers (Becton-Dickinson Immunocytometry Systems, San Jose, Calif.) equipped with argon ion lasers. Dulbecco's phosphate buffered saline (Irvine Scientific, Santa Anna, Calif.) was used as sheath fluid. Fluorescence emitted by each stained sperm after laser excitation (100 mW UV) was directed through a 400 nm long pass filter to forward (0°) and right angle (90°) detectors. Properly oriented sperm were gated based on lower (YSORT®) fluorescence intensity, and the sorted sperm were collected. In other words, apparatus was configured to select sperm with a sperm karyotic index of about 100. Sperm were analyzed at a rate of 3,000-3,500 cells per second and sorted sperm were collected at a rate of 20-25 cells per second. Sperm used for IUI were sorted into TYB refrigeration medium (Irvine Scientific, Santa Ana, Calif.) or, if the patient was egg yolk- or antibiotic sensitive, into BWW+10% BA or Modified Hams F01 (Irvine Scientific, Santa Ana, Calif.).

At the conclusion of sorting, collected sperm were centrifuged to concentrate recovered cells in a final volume of 400 μl. Post-sort motility and progression were evaluated at 35° C. under paraffin oil using Hoffman illumination. The sorted specimens were evaluated for the degree of enrichment euploid sperm (post-sort purity) using fluorescence in situ hybridization (FISH).

### EXAMPLE 3

Fluorescence In Situ Hybridization (FISH).

The FISH procedure utilizes the following reagents: Carnoy's Fixative (3:1 Methanol/Acetic Acid); 2×SSC; 4× SSC, pH 7; 0.4× SSC/0.3% Igepal, pH 7; P/N Buffer, pH 8 (Solution A: Deionized water 1800 ml; Na₂HPO₄ 28.4 g; Igepal 2.0 ml and Solution B: Deionized water 200 ml; NaH₂PO₄ 2.76 g; Igepal 200 ul). P/N Buffer is made by bringing both solutions A and B to room temperature before measuring pH. 500 ml of Solution A is placed in a bottle the pH is adjusted to 8.0 using Solution B (usually about 20 ml).

The 13q telomere probe was obtained from Cytocell, Ltd. (UK) lot #040321-011 and the 14q telomere probe was obtained from Vysis, Inc. (Downers Grove, Ill.). lot #53747. The FISH procedure was a modification (Pieters et al., Cytogenet. Cell Genet., 53, 15-19; 1990) of the one-DNA probe standard protocol as previously described (Fugger et al., Hum. Reprod. 30, 2367-70; 1998).

Sperm samples and control sperm are removed from −20° C. storage and thawed at room temperature. About 0.9 microliters of sample is spread in the designated octant using a P-2 pipettor and allowed to air dry.

The sperm are fixed by adding 1 microliter Carnoy's fixative to each octant using a clean pipette tip for each octant and allowing the slide to completely air dry. 4 microliters HPLC water are added to the first octant using a pipette and immediately aspirated off. The slide is then again allowed to dry and then evaluated under a phase microscope to verify the removal of proteins. An additional 2 microliters Carnoy's fixative is added to each octant and each slide is then dried on a 37° C. slide warmer for 1 minute. Next, 5 microliters fixative is added to each octant and the slide is again allowed to dry slide on a 37° C. slide warmer for 1 minute.

During pretreatment, a 15 ml conical tube filled with 2×SSC is preheated for 10 minutes in a 37° C. waterbath. Next, warm 2×SSC is added to each octant while heating the slide on a slide warmer. The solution is aspirated and the procedure repeated twice. After the last wash, the slide is shaken off and allowed to dry.

The slides are blotted before DTT treatment. DTT is mixed with 38 ml of Tris-HCl. The 50 mM DTT/Tris-HCl solution is placed in a coplin jar and the slides are treated in the mixture for 10 minutes at room temperature on the rotator. The slides are then washed three times in 2×SSC at room temperature, for 2 minutes each.

For the hybridization procedure, a ThermoBrite hybridization machine is used. First, the blank slides are removed from the heating surface of the machine and wiped of any rubber cement or debris from the surface using a damp paper towel. The machine is programmed to a melting phase at 75° C. for 5 minutes and hybridization phase at 37° C. for 20 hours. The appropriate amount of probe mix is added to each area of the slide and coversliped with the appropriate sized coverslip, ensuring the cells are within the coverslip boundaries. Generally, 24×50 mm coverslip are used for 8 cells, 22×22 mm coverslip for 4 cells, and 11×22 mm coverslip for 2 cells. The coverslips are completely sealed with rubber cement.

During the post-hybridization wash, the slides are washed with 0.4×SSC/0.3% Igepal at 72-73° C. for 5 minutes. Next, the slides are placed into a polypropylene coplin jar with 50 ml of 4×SSC, pH 7 and incubated for 1 minute at room temperature. The slides are then washed with 40-50 ml of DPBS and incubated at 1 minutes at room temperature. Then the slides are washed with about 50 ml of room temperature P/N buffer and incubated 1 minutes at room temperature. 10-20 microliters of Antifade II is applied to the slide in the etched area and the slides are coversliped with a 24×50 mm coverslip.

### EXAMPLE 4

Sperm Scoring Criteria The following outlines the criteria and procedures for analyzing sperm samples for fluorescence in situ hybridization. Specimen criteria are necessary to define so that technicians can differentiate between acceptable and unacceptable samples to minimize scoring errors. The following types of samples are considered unacceptable and are not be scored: Sperm head that is broken or missing nuclear material; Sperm head containing no signals; Sperm head that is scratched or damaged; Fluorescent background is indistinguishable from the actual signals within the sperm head; The signals are obscured by cytoplasm or debris covering the sperm head; Overlapping sperm heads; Sperm heads which are vastly larger or smaller than the typical sperm head observed on the slide; Sperm head which is amorphous in shape; Sperm head which is tetraploid for the targeted chromosomes.

The following types of samples are considered sub-optimal but may be scored with a reasonable degree of confidence: Sperm head that is covered with cytoplasm, but the signals can be visualized; Sperm head which has significant fluorescent background present, however, the true signals are distinguishable from background fluorescence.

The following types of samples are considered optimal for scoring: Sperm head with clearly defined borders; Sperm head with a round, flat appearance; Sperm head with clear readable signals for each chromosome locus targeted; Sperm head with little or no background fluorescence; Sperm head that is clear of cytoplasm and debris.

Analyzing sperm samples for FISH: The sperm sample is visualized and focused under 60× magnification using a triple band pass cube. Before scoring, the sample slide is scanned for the area which displays the best hybridization at first observation. All sperm heads in the designated area are scored with the aforementioned exceptions. The different fluors are then analyzed under the single band pass cubes and emitters. Preferably, two qualified technologists individually analyze the sperm sample for the chromosome regions targeted by fluorescence in situ hybridization. Each technologist scores 100 sperm heads from different region of the probed area on the slide.

The following are the most common sources of scoring errors and guidelines for minimizing errors when these situations are encountered:

Signal bleed-through—this is defined as a fluor that is visible under a cube/emitter specific for a different fluor, for example: Probes which are labeled with blue are occasionally visible under the aqua single band pass cube and vice versa for probes labeled with aqua. Additionally, probes that are labeled with orange will bleed through into view under the gold cube, probes labeled with orange into the red filter and vice versa. Occasionally, very strong probes that are labeled with gold will bleed through into view under the green filter. To count the correct number of signals for each of these fluors, one should: Examine the fluors using the filter where both fluors are visible and/or Switch to the cube/emitter which displays only one of the fluors and notice which signals are still visible. These signals are the true fluors for the single band pass cube in use. The signals that disappeared are the true fluors for the single band pass cube in which both fluors were visible.

Cross hybridization—Occasionally, the probes will cross hybridize to other non-targeted regions of DNA causing additional fluorescent signals within the sperm head. To distinguish cross-hybridization signals from true signals, compare the intensity of all fluorescent domains. True signals should have a higher fluorescent intensity than cross-hybridized signals.

Backgroundfluorescence—occasionally unspecific background (pieces of cellular material, sperm tail or debris) within the sperm head will fluoresce. Depending on the source of the background the true signals can be distinguished using the following guidelines: A fluorescent domain on a vastly different plane of focus than other fluorescent signals and the sperm head is not a true signal. A fluorescent domain that is very pinpoint and shiny in comparison with the other signals is not a true signal. A fluorescent domain that is visible using all single band pass filters is not a true signal. A fluorescent domain that much less intense than other fluorescent signals of the same fluor is not a true signal.

Signal overlap—occasionally, two or more signals may appear to be in the exact same location within the sperm head or overlapping when looking through the triple band pass filter. If the overlapping signals are different colors, the signals can usually be easily distinguished from each other when the individual band pass filters are used to visualize each fluor separately. If the overlapping signals are the same color, the following rules are used to determine the number of targets present: Two signals that are more than one domain apart are counted as two distinct targets. Signals less than one domain apart are counted as two distinct targets if they are on two different planes of focus. Blue and aqua are more challenging because the wavelengths are so similar and the signals frequently “bleed-through” into each other's single band pass filters. When an overlap occurs involving two bleed-through fluors, the following guidelines are used: Examine the fluors using the filter where both fluors are visible. Carefully view the area with the suspected overlap. Switch to the cube which displays only one of the fluors. If the shape and/or size of the signal changes (i.e., the other fluor disappears from view), there are two overlapping signals in the area.

Split signals—One fluorescent domain may appear as two individual signals. This is due to chromatids splitting, thus making one hybridized locus appear as two hybridized domains. The following guidelines are used to differentiate between two individual signals and chromatid splits: Signals must be at least one domain apart to score the signals as two distinct targets, except, if the signals are one domain apart but one signal is much weaker than the other, the signals are counted as one. If the signals are one domain apart but there is contact between the signals.

Large, diffuse signals—this is most common with probes that hybridize to highly repetitive regions of DNA, such as CEP probes. If signals appear to be large, diffuse, and/or spread out, the signal will be scored as one distinct target only if the target is a centromeric repetitive region of DNA. Additionally, unequal repetitive regions of DNA or polymorphic regions can produce fluors differing in size between two homologous chromosomes.

### EXAMPLE 5

Enriching a Sperm Sample for Euploid Sperm

A Roberstonian translocation career volunteer's (Patient A) sperm was tested by FISH using sub-telomeric probes that localize to the ends of the chromosomes involved in his translocation to determine the percentage of abnormal gametes in his ejaculate using the techniques described in the Examples above.

In Patient A's raw sample, 73 of the 216 cells counted were scored as abnormal (unbalanced), leaving a 33.8% normal count.

Patient A's sperm was then sorted in two batches (Sort A and B) using the methodology outlined in Example 2. In Patient A's Sort A sample, 45(22.5%) of the 200 cells counted were scored as abnormal (unbalanced), leaving a 77.5% normal count. In Patient A's Sort B sample, 44 (22.0%) of the 200 cells counted were scored as abnormal (unbalanced), leaving a 78% normal count. Table 2. In normal control sperm, 211 (93.4%) of the 226 cells counted were found to be normal (expected count of FISH probes). The results indicate that after sorting Patient A's sperm resulted in an about 34% decrease in the percentage of abnormal sperm compared to his raw sample. This demonstrates that the inventive methods greatly enhance the likelihood that Patient A will be capable of fathering a genetically healthy baby.

TABLE 2

| | # Nuclei | | | % |
|---|---|---|---|---|
| Probe | Scored | Normal | Abnormal | Abnormal |
| Control | 226 | 211 | 15 | 7.5 |
| 762 lot | | | | |
| 110 | | | | |
| Patient: | 216 | 143 | 73 | 33.8 |
| Raw sperm | | | | |
| Patient: | 0 | 0 | 0 | — |
| Prep sperm | | | | |
| Patient: | 200 | 155 | 45 | 22.5 |
| Sort A | | | | |
| Patient: | 200 | 156 | 44 | 22.0 |
| Sort B | | | | |

In this disclosure there are described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

What is claimed is:

1. A method of enriching a sperm sample for euploid sperm comprising:
a) obtaining a sperm sample comprising sperm cells from a mammalian male;
b) contacting the sperm cells with a detectable DNA-interacting agent that imparts a sperm cell with a signal intensity proportional to the amount of DNA present in the sperm cell;
c) separating those sperm cells that have a signal intensity indicative of euploid sperm from those that have a signal intensity indicative of aneuploid sperm; and
d) collecting said separated sperm cells to obtain an enriched sperm sample.

2. The method of claim 1 wherein said agent is Hoechst 33342.

3. The method of claim 1 wherein said separating is carried out by flow cytometery.

4. The method of claim 3 wherein said flow cytometery is fluorescence activated cell sorting.

5. The method of claim 1 wherein the signal intensity indicative of euploid sperm corresponds to a sperm karyotype index between about 99.5 and about 103;
wherein a sperm karyotype index of 100 is defined as the signal intensity associated with normal Y-bearing sperm and a sperm karyotype index of 102.8 is defined as the signal intensity associated with normal X-bearing sperm.

6. The method of claim 5 wherein the signal intensity indicative of euploid sperm corresponds to a sperm karyotype index of about 100.

7. The method of claim 6 wherein the sperm is human sperm.

8. The method of claim 1 wherein the signal intensity indicative of euploid sperm corresponds to a sperm karyotype index between about 99.5 and about 104;
wherein a sperm karyotype index of 100 is defined as the signal intensity associated with normal Y-bearing sperm and a sperm karyotype index of 103.4 is defined as the signal intensity associated with normal X-bearing sperm.

9. The method of claim 8 wherein the signal intensity indicative of euploid sperm corresponds to a sperm karyotype index of about 100.

10. The method of claim 9 wherein the sperm is boar sperm.

11. The method of claim 1 wherein the signal intensity indicative of euploid sperm corresponds to a sperm karyotype index between about 99.5 and about 104;
wherein a sperm karyotype index of 100 is defined as the signal intensity associated with normal Y-bearing sperm and a sperm karyotype index of 103.8 is defined as the signal intensity associated with normal X-bearing sperm.

12. The method of claim 11 wherein the signal intensity indicative of euploid sperm corresponds to a sperm karyotype index of about 100.

13. The method of claim 12 wherein the sperm is bull sperm.

14. The method of claim 1 wherein the signal intensity indicative of euploid sperm corresponds to a sperm karyotype index between about 99.5 and about 104.5;
wherein a sperm karyotype index of 100 is defined as the signal intensity associated with normal Y-bearing sperm and a sperm karyotype index of 104.2 is defined as the signal intensity associated with normal X-bearing sperm.

15. The method of claim 14 wherein the signal intensity indicative of euploid sperm corresponds to a sperm karyotype index of about 100.

16. The method of claim 15 wherein the sperm is ram sperm.

17. The method of claim 1 wherein the sperm sample and the enriched sperm sample are analyzed by fluorescence in situ hybridization (FISH) for chromosomal abnormalities.

18. The method of claim 1 wherein said male produces sperm that contain structural chromosomal abnormalities

19. The method of claim 1, wherein aneuploid sperm have numerical chromosomal abnormalities.

20. The method of claim 1 where said male is a translocation carrier.

21. The method of claim 1 wherein said male is selected from the group consisting of a human, a bull, a horse, a pig, a goat, and a boar.

22. The method of claim 20 wherein said male is a Robertsonian translocation carrier.
